(19) United States
(12) Patent Application Publication
(10) Pub. No.: US 2014/0206857 A1
(43) Pub. Date: Jul. 24, 2014

(54) METHOD FOR THE PREPARATION OF 2-(4-METHOXYCARBONYLPYRAZOL-1-YL)ADENOSINE AND 2-(4-ETHOXYCARBONYLPYRAZOL-1-YL)ADENOSINE

(71) Applicants: Lubomir Kvapil, Slatinice (CZ); Pavel Hradil, Hlusovice (CZ); Martin Grepl, Hlusovice (CZ); Petr Slezar, Olomouc (CZ); Barbora Dvorakova, Most 1 (CZ)

(72) Inventors: Lubomir Kvapil, Slatinice (CZ); Pavel Hradil, Hlusovice (CZ); Martin Grepl, Hlusovice (CZ); Petr Slezar, Olomouc (CZ); Barbora Dvorakova, Most 1 (CZ)

(21) Appl. No.: 14/239,261

(22) PCT Filed: Aug. 9, 2012
(86) PCT No.: PCT/CZ2012/000078

§ 371 Date: Feb. 18, 2014

(30) Foreign Application Priority Data
Aug. 18, 2011 (CZ) PV 2011-511

(51) Int. Cl. C07H 19/16; C07H 1/00

(52) U.S. Cl. CPC C07H 19/16; C07H 1/00
USPC 536/27.61

(57) ABSTRACT

A method for the preparation of 2-(4-methoxycarbonylpyrazol-1-yl)adenosine of formula 1a and 2-(4-ethoxycarbonylpyrazol-1-yl)adenosine of formula 1b by reaction of 2-hydrazinoadenosine of formula III and the sodium salt of 3,3-dimethoxy-2-methoxycarbonylpropen-1-ol of formula Va or the sodium salt of 3,3-diethoxy-2-ethoxycarbonylpropen-1-ol of formula Vb in combination with a solvent and an acidic agent.

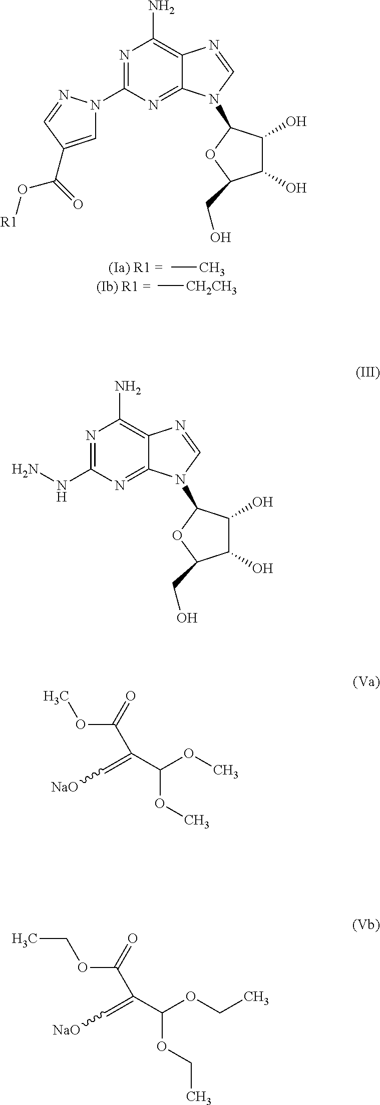

### TECHNICAL FIELD

The invention relates to a new method for the preparation of 2-(4-methoxycarbonylpyrazol-1-yl)adenosine of formula Ia and 2-(4-ethoxycarbonylpyrazol-1-yl)adenosine of formula Ib

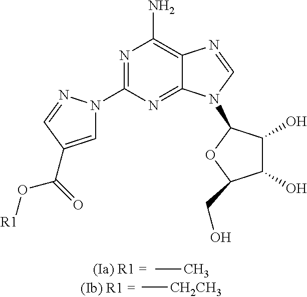

wherein 2-(4-ethoxycarbonylpyrazol-1-yl)adenosine of formula Ib is an intermediate used for the manufacture of Regadenoson of formula II

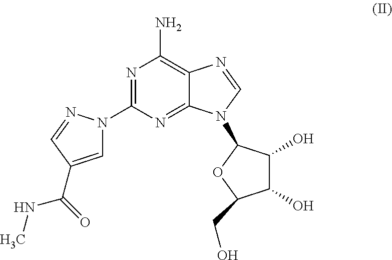 [SMILES: CNC(=O)c1cnn(-c2nc(N)c3ncn([C@@H]4O[C@H](CO)[C@@H](O)[C@H]4O)c3n2)c1]

and 2-(4-methoxycarbonylpyrazol-1-yl)adenosine of formula Ia is a newly prepared compound, which is a potential intermediate for the manufacture of Regadenoson of formula II.

Regadenoson is used as a coronary vasodilator for diagnostic purposes during radionuclide examinations of the heart.

### BACKGROUND ART

The methods for the preparation of 2-(4-ethoxycarbonylpyrazol-1-yl)adenosine of formula Ib that are known so far are based on condensation of 2-hydrazinoadenosine of formula III

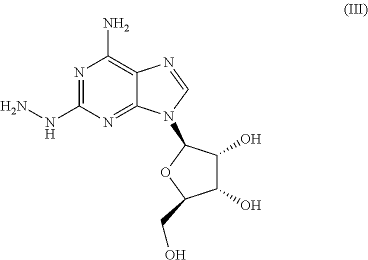 [SMILES: NNc1nc(N)c2ncn([C@@H]3O[C@H](CO)[C@@H](O)[C@H]3O)c2n1]

and (ethoxycarbonyl)malondialdehyde of formula IV.

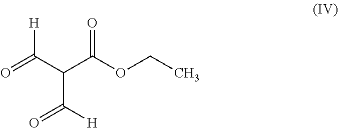

A reaction of 2-hydrazinodenosine of formula III and (ethoxycarbonyl)malondialdehyde of formula IV in a methanol/acetic acid mixture under boiling for 3 hours with the yield of 2-(4-ethoxycarbonylpyrazol-1-yl)adenosine of formula Ib of 91% is known (U.S. Pat. No. 6,403,567 or J. Zablocki et al.—Nucleotides, Nucleosides and Nucleic Acid 2001, 20 (4-7), 343-360).

Another well-known procedure uses a reaction of 2-hydrazinoadenosine of formula III and (ethoxycarbonyl)malondialdehyde of formula IV in isopropyl alcohol under boiling for 4 hours; the reported yield of 2-(4-ethoxycarbonylpyrazol-1-yl)adenosine of formula Ib is 89.6% and its purity is 96.6% (HPLC) (WO 2007/092372 and WO 2008/143667).

Another similar procedure to carry out this reaction under boiling in ethanol is described in patent application US 2010/0267953.

Literature also mentions the possibility of synthesis of derivatives of the ester Ib by means of a cross-coupling reaction between 2-iodoadenosine and derivatives of 4-pyrazole carboxylic acid (Drugs of the Future 2004, 29 (10), 998 and in U.S. Pat. No. 6,514,949). However, this synthesis is not sufficiently documented with experimental data, but what can be assumed is that complexes with heavy metals are used in this case and the synthesized derivative has then to be laboriously (chromatographically) purified.

A disadvantage of the above mentioned methods consists in a low purity of the prepared ester Ib due to relatively harsh reaction conditions (boiling of the reaction mixture for several hours) and also with regard to a limited stability of the starting (ethoxycarbonyl) malondialdehyde of formula IV (see e.g. S. H. Bertz—J. Org. Chem. 1982, 47, 2216) and especially of 2-hydrazinoadenosine of formula III, which is very sensitive to heat (see e.g. H. J. Schaeffer et al.—J. Am. Chem. Soc. 1958, 80, 3738).

### DISCLOSURE OF INVENTION

The above mentioned disadvantages are overcome by the process of the present invention, which consists in a method for the preparation of 2-(4-methoxycarbonylpyrazol-1-yl)adenosine of formula Ia and 2-(4-ethoxycarbonylpyrazol-1-yl)adenosine of formula Ib

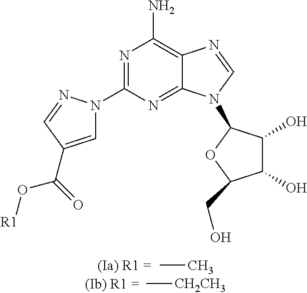

by reaction of 2-hydrazinoadenosine of formula (III)

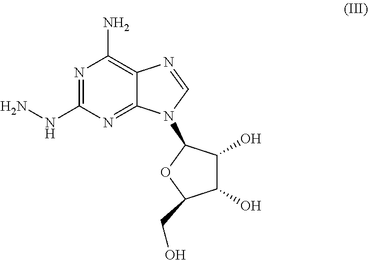 [SMILES: NNc1nc(N)c2ncn([C@@H]3O[C@H](CO)[C@@H](O)[C@H]3O)c2n1]

wherein the sodium salt of 3,3-dimethoxy-2-methoxycarbonylpropen-1-ol of formula Va in case of 2-(4-methoxycarbonylpyrazol-1-yl)adenosine of formula Ia or the sodium salt of 3,3-diethoxy-2-ethoxycarbonylpropen-1-ol of formula Vb in case of 2-(4-ethoxycarbonylpyrazol-1-yl)adenosine of formula Ib is used for the reaction.

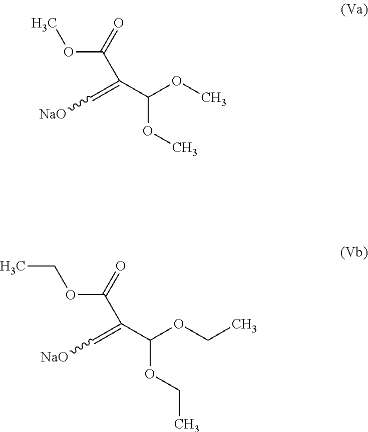

It is an object of the invention that the reaction is carried out in combination with a solvent and in combination with an acidic agent.

It is another object of the invention that water or a solvent from the group of alcohols, especially methanol, ethanol and isopropyl alcohol, or a solvent from the group of polar aprotic solvents, especially dimethyl sulphoxide, is used as the solvent for the reaction or their mutual mixtures are used as said solvent.

It is still another object of the invention that the reaction is carried out in combination with an acidic agent, wherein preferably an organic acid, especially a carboxylic acid such as formic, acetic and propionic acid, is used as the acidic agent.

It is further possible to use, as the acidic agent, a mineral acid, especially hydrochloric, sulphuric and phosphoric acid. Acidic salts such as disulphites, hydrogen sulphates and dihydrogen phosphates can also be used as the acidic agent.

It has been proved that without the use of an acidic agent the reaction proceeds with remarkably lower yield and purity.

It is another object of the invention that the reaction is carried out with excess of the sodium salt of 3,3-dimethoxy-2-methoxycarbonylpropen-1-ol of formula Va or of the sodium salt of 3,3-diethoxy-2-ethoxycarbonylpropen-1-ol of formula Vb at the temperature of 25 to 60° C. for 2 to 7 hours.

The sodium salt of 3,3-dimethoxy-2-methoxycarbonylpropen-1-ol of formula Va or the sodium salt of 3,3-diethoxy-2-ethoxycarbonylpropen-1-ol of formula Vb can be easily prepared by the Claisen condensation of methyl 3,3-dimethoxypropionate of formula VIa or ethyl 3,3-diethoxypropionate of formula VIb

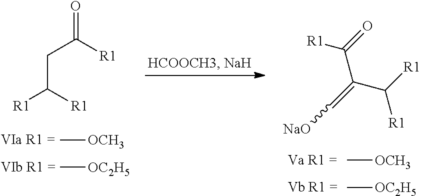

with methyl or ethyl formate using a strong base such as sodium hydride (see e.g. P. Zhichkin et al., Synthesis 2002, No 6, 720)

The above mentioned salts are relatively stable and well available, for example, the sodium salt of 3,3-dimethoxy-2-methoxycarbonyl-propen-1-ol of formula Va is commercially available in the solid state. Both the salts can also be used in the form of a reaction mixture directly without isolation as a solution or suspension.

In the published patent U.S. Pat. No. 6,403,567 there was achieved a yield of 91% without specifying the purity and in the patent application WO 2007/092372 there was achieved a yield of 89.6% at the purity of 96.6%. These methods have been verified and the yields and purity of 2-(4-ethoxycarbonylpyrazol-1-yl)adenosine of formula Ib prepared in accordance with these documents were compared to the yields and purity of 2-(4-methoxycarbonylpyrazol-1-yl)adenosine of formula Ia prepared in accordance with the present invention. The results are summarized in Table 1.

The same starting 2-hydrazinoadenosine with the HPLC purity of 99.2% was used for all the experiments.

TABLE 1

| | Yield /%/ | HPLC purity /%/ |
|---|---|---|
| U.S. Pat. No. 6,403,567 | 85.3 | 93.9 |
| WO 2007/092 372 | 80.0 | 92.7 |
| Present invention, Example 1 | 95.0 | 99.2 |

The table shows that all the three methods provided high yields.

The methods in accordance with U.S. Pat. No. 6,403,567 and WO 2007/092372 achieved yields in the range of 80 to 85%. However, the method according to the present invention achieved a higher yield, namely 95%.

An even more significant difference was achieved regarding the purity of the prepared compounds. The purity obtained in accordance with U.S. Pat. No. 6,403,567 and WO 2007/092372 was approximately 93% (HPLC), but only in the procedure according to the present invention an HPLC purity over 99% was achieved. And it is purity that is one of the most important parameters of products and intermediates in pharmaceutical industry (see e.g. the ICH Harmonized Tripartite Guideline, Impurities in New Drug Substances Q3A(R2), 2006). This means that it is only the product in accordance with the present invention that does not require re-purification e.g. by crystallization, while the two products prepared in accordance with U.S. Pat. No. 6,403,567 and WO 2007/092372 will additionally need certain re-purification.

The advantages of the method according to the invention are as follows:
The reaction temperature is moderate: 25 to 60° C.
The reaction time is short: 2 to 7 hours.
Under optimum conditions the product purity is considerably higher (over 99%, HPLC) than described so far (96.6%, HPLC in accordance with PCT WO 2007/092372); the purity can be further easily increased by re-crystallization e.g. using a dimethyl sulfoxide/methanol mixture.

### EXAMPLES

The essence of the method according to the invention is clarified in more detail in the following examples.

These examples only have an illustrative character and do no limit the scope of the invention in any way.

NMR spectra were measured using a Varian NMR 400 device at 400 MHz (¹H) and at 100 MHz (¹³C). The samples were dissolved in 15 mg/0.7 ml of DMSO-d₆ and measured at the temperature of 300 K.

Differential Scanning calorimetry (DSC) was measured using Perkin Elmer instrumentation, the Pyris Diamond DSC model with evaluation using the Pyris software, version 5.0. The samples were analyzed in open aluminium pans in a nitrogen atmosphere.

### Example 1

10 ml of acetic acid are added to a suspension of 8.8 g of 2-hydrazinoadenosine (29.6 mmol) in 60 ml of water and 20 ml of methanol. After stirring at the laboratory temperature for ca. 5 mins, a solution is produced, to which 7.6 g of the sodium salt of 3,3-dimethoxy-2-methoxycarbonylpropen-1-ol (38.5 mmol) are added and, after stirring for another ca. 5 mins, a yellow reaction solution is obtained, which is heated to 50 to 55° C. and maintained for 2 hours while the product precipitates. Then the thick reaction mixture is cooled and filtered. After filtration and washing with water and methanol the product is dried to dryness in vacuo. This procedure provides 11.0 g of 2-(4-methoxycarbonylpyrazol-1-yl)adenosine, i.e. 95.0%, with the purity of 99.2% (HPLC).

An analytically pure sample is obtained by re-crystallization from a dimethyl sulphoxide/methanol mixture.

Melting point—uncorrected: 225-228° C.

The Differential Scanning calorimetry (DSC) exhibits an endothermic transition at 227.6° C.

¹H NMR (DMSO-d₆, 400 MHz) δ 8.94 (1H, d, J=0.7 Hz), 8.41 (1H, s), 8.11 (1H, d, J=0.7 Hz), 7.83 (2H, s), 5.92 (1H, d, J=6.2 Hz), 5.49 (1H, d, J=6.5 Hz), 5.21 (1H, d, J=4.8 Hz), 5.00 (1H, t, J=5.7 Hz), 4.59 (1H, m), 4.16 (1H, m), 3.95 (1H, m), 3.79 (3H, s), 3.63 (2H, m)

¹³C NMR (DMSO-d₆, 100 MHz) δ 162.85, 156.87, 150.56, 150.52, 142.54, 140.71, 132.51, 118.60, 115.91, 87.66, 86.13, 74.11, 70.87, 61.87, 51.99

### Example 2

10 ml of formic acid are added to a suspension of 8.8 g of 2-hydrazinoadenosine (29.6 mmol) in 40 ml of water and 40 ml of dimethyl sulphoxide. After stirring at the laboratory temperature for ca. 5 mins, a solution is produced, to which 7.6 g of the sodium salt of 3,3-dimethoxy-2-methoxycarbonylpropen-1-ol (38.5 mmol) are added and, after stirring for another ca. 5 mins, a yellow reaction solution is obtained, which is heated to 45 to 50° C. and maintained for 3 hours, while the product precipitates. Then the thick reaction mixture is cooled and filtered. After filtration and washing with water and methanol the product is dried to dryness in vacuo.

This procedure provides 11.0 g of 2-(4-methoxycarbonylpyrazol-1-yl)adenosine, i.e. 95.0%, with the purity of 98.7% (HPLC).

### Example 3

10 ml of propionic acid are added to a suspension of 8.8 g 2-hydrazinoadenosine (29.6 mmol) in 60 ml of water and 20 ml of isopropyl alcohol. After stirring at the laboratory temperature for ca. 5 mins, a solution is produced, to which 7.6 g of the sodium salt of 3,3-dimethoxy-2-methoxycarbonylpropen-1-ol (38.5 mmol) are added and, after stirring for another ca. 5 mins, a yellow reaction solution is obtained that is stirred at 25° C. for 7 hours, while the product precipitates. Then the thick reaction mixture is cooled and filtered. After filtration and washing with water and methanol the product is dried to dryness in vacuo.

This procedure provides 10.7 g of 2-(4-methoxycarbonylpyrazol-1-yl)adenosine, i.e. 92.0%, with the purity of 98.5% (HPLC).

### Example 4

15 ml of acetic acid are added to a suspension of 8.8 g of 2-hydrazinoadenosine (29.6 mmol) in 40 ml of water. After stirring at the laboratory temperature for ca. 5 mins, a solution is produced, to which a solution of 9.96 g of the sodium salt of 3,3-diethoxy-2-methoxycarbonylpropen-1-ol in 1,2-dimethoxyethane is added. A yellow reaction solution is obtained, which is heated to 55 to 60° C. and maintained for 3 hours, while the product precipitates. Then the thick reaction mixture is cooled and filtered. After filtration and washing with water and methanol the product is dried to dryness in vacuo. This procedure provides 10.9 g of 2-(4-methoxycarbonylpyrazol-1-yl)adenosine, i.e. 91.0%, with the purity of 98.0% (HPLC).

### Example 5

6 ml of acetic acid are added to a suspension of 8.8 g 2-hydrazinoadenosine (29.6 mmol) in 50 ml of water. After stirring at the laboratory temperature for ca. 5 mins, a solution is produced, which is poured to the reaction mixture containing 7.5 g of the sodium salt of 3,3-dimethoxy-2-methoxycarbonylpropen-1-ol (37.8 mmol) in 45 ml of 1,2-dimethoxyethane (prepared according to P. Zhichkin et al., Synthesis 2002, No 6, 720). A reaction solution is obtained, which is heated to 55° C. and maintained for 3 hours, while the product precipitates. Then the thick reaction mixture is cooled and filtered. After filtration and washing with water and methanol the product is dried to dryness in vacuo. This procedure provides 10.6 g of 2-(4-methoxycarbonylpyrazol-1-yl)adenosine, i.e. 91.5%, with the purity of 99.0% (HPLC).

### Example 6

3.3 ml of hydrochloric acid are added to a suspension of 8.8 g 2-hydrazinoadenosine (29.6 mmol) in 100 ml of water. 7.5 g of the sodium salt of 3,3-dimethoxy-2-methoxycarbonylpropen-1-olu are added to the resulting solution. The obtained solution is heated to 40° C. until the starting 2-hydrazinoadenosine has completely reacted (HPLC check), for about 4 hours. Then the reaction mixture is cooled to the laboratory temperature and the solids are removed by filtration, washed with water and methanol and dried in vacuo. This procedure provides 10.9 g of 2-(4-methoxycarbonylpyrazol-1-yl)adenosine, i.e. 91.0%, with the purity of 97.5% (HPLC).

Phosphoric acid or sulphuric acid, for example, can be used instead of hydrochloric acid under similar conditions.

### Example 7

0.3 g of sodium disulphite and 7.6 g of the sodium salt of 3,3-dimethoxy-2-methoxycarbonylpropen-1-ol (38.5 mmol) are gradually added to a suspension of 8.8 g 2-hydrazinoadenosine (29.6 mmol) in 100 ml of water. Being stirred and heated the suspension converts into a solution, which is heated to the temperature of 60° C. for 5 hours, while the product precipitates. Then the thick reaction mixture is cooled and filtered. After filtration and washing with water and methanol the product is dried to dryness in vacuo. This procedure provides 10.6 g of 2-(4-methoxycarbonylpyrazol-1-yl)adenosine, i.e. 91.5%, with the purity of 99.1% (HPLC).

Potassium hydrogen sulphate or sodium dihydrogen phosphate, for example, can be used instead of sodium disulphite under similar conditions.

1. A method for the preparation of 2-(4-methoxycarbonylpyrazol-1-yl)adenosine of formula Ia and 2-(4-ethoxycarbonylpyrazol-1-yl)adenosine of formula Ib 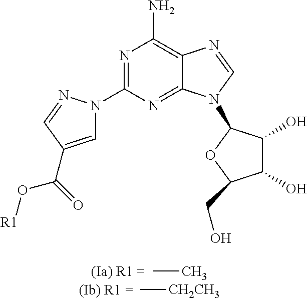
by reaction of 2-hydrazinoadenosine of formula III, 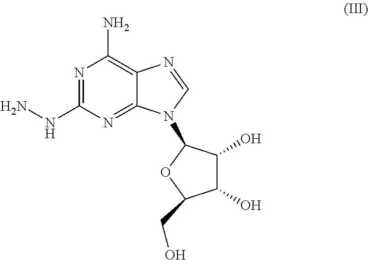 [SMILES: NNc1nc(N)c2ncn([C@@H]3O[C@H](CO)[C@@H](O)[C@H]3O)c2n1]
wherein the reaction is carried out using the sodium salt of 3,3-dimethoxy-2-methoxycarbonylpropen-1-ol of formula Va in the case of 2-(4-methoxycarbonylpyrazol-1-yl)adenosine of formula Ia or the sodium salt of 3,3-diethoxy-2-ethoxycarbonylpropen-1-ol of formula Vb in the case of 2-(4-ethoxycarbonylpyrazol-1-yl)adenosine of formula Ib 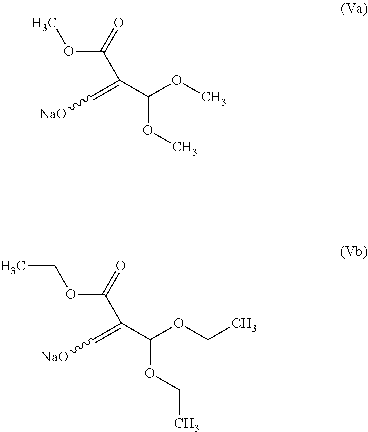
in the presence of a solvent and an acidic agent.

2. The method according to claim 1, wherein the acidic agent is formic, acetic or propionic acid.

3. The method according to claim 1, wherein the acidic agent is hydrochloric, sulphuric or phosphoric acid.

4. The method according to claim 1, wherein the acidic agent is disulphites, hydrogen sulphates or dihydrogen phosphates.

5. The method according to claim 1, wherein the solvent is water or a solvent from the group of alcohols or a solvent from the group of polar aprotic solvents or their mutual mixtures.

6. The method according to claim 1, wherein the reaction is carried out for 2 to 7 hours.

7. The method according to claim 1, wherein the reaction is carried out at the temperatures of 25 to 60° C.

8. 2-(4-Methoxycarbonylpyrazol-1-yl)adenosine of formula Ia, characterized by:
Melting point—uncorrected: 225-228° C.
The Differential Scanning calorimetry DSC exhibits an endothermic transition at 227.6° C.
¹H NMR (DMSO-d₆, 400 MHz) δ 8.94 (1H, d, J=0.7 Hz), 8.41 (1H, s), 8.11 (1H, d, J=0.7 Hz), 7.83 (2H, s), 5.92 (1H, d, J=6, 2 Hz), 5.49 (1H, d, J=6.5 Hz), 5.21 (1H, d, J=4.8 Hz), 5.00 (1H, t, J=5.7 Hz), 4.59 (1H, m), 4.16 (1H, m), 3.95 (1H, m), 3.79 (3H, s), 3.63 (2H, m)
¹³C NMR (DMSO-d₆, 100 MHz) δ 162.85, 156.87, 150.56, 150.52, 142.54, 140.71, 132.51, 118.60, 115.91, 87.66, 86.13, 74.11, 70.87, 61.87, 51.99.
